(19) United States
(12) Patent Application Publication
(10) Pub. No.: US 2026/0048109 A1
(43) Pub. Date: Feb. 19, 2026

(54) Methods for Vaccinating against Salmonella Species

(71) Applicant: University of Maryland, Baltimore, Baltimore, MD (US)

(72) Inventors: Alan S. Cross, Chevy Chase, MD (US); Scott M. Baliban, Washington, DC (US)

(73) Assignee: University of Maryland, Baltimore, Baltimore, MD (US)

(21) Appl. No.: 19/299,526

(22) Filed: Aug. 14, 2025

Related U.S. Application Data
(60) Provisional application No. 63/682,964, filed on Aug. 14, 2024.

(51) Int. Cl. A61K 39/112; A61K 39/00; A61K 39/108; A61P 31/04

(52) U.S. Cl. CPC A61K 39/0275; A61K 39/0258; A61P 31/04; A61K 2039/522; A61K 2039/575

(57) ABSTRACT

Provided herein are methods for vaccinating a subject against a Salmonella species, including typhoidal and non-typhoidal Salmonella species and serovars thereof with an Escherichia coli J5 dLOS/OMP vaccine. The vaccine is cross-reactive with the lipopolysaccharide conserved inner core region of the Salmonella species and cross-protective with the non-typhoidal Salmonella species. Vaccination reduces the likelihood of or prevents the onset of a salmonella infection. Also provided is a method of immunizing against antimicrobial resistant Salmonella species by complementing the J5 dLOS/OMP vaccine with a Salmonella species-specific vaccine.

### CROSS-REFERENCE TO RELATED APPLICATIONS

This non-provisional patent application claims benefit of priority under 35 U.S.C. § 119 (e) of provisional application U.S. Ser. No. 63/682,964, filed Aug. 14, 2024, the entirety of which is hereby incorporated by reference.

### STATEMENT OF GOVERNMENT SUPPORT

This invention was made with government support under Grant Numbers AI135561 and AI168587 awarded by the National Institutes of Health. The government has certain rights in the invention.

### BACKGROUND OF THE INVENTION

### Field of the Invention

The present invention relates to the fields of medicine and vaccines. More particularly, the present invention relates to a cross-reactive vaccine that targets the highly conserved inner core region of lipopolysaccharide and elicits a cross-reaction against Salmonella spp.

### Description of the Related Art

Salmonella enterica subspecies enterica is a Gram-negative bacterium (GNB) with over 2,600 serovars, many of which can cause disease in humans and animals. The typhoidal serovars Typhi and Paratyphi A, B, and C are the etiological agents of enteric (typhoid and paratyphoid) fever, a significant public health problem in resource limited particularly in Africa and Asia (1-3).

In sub-Saharan Africa, nontyphoidal Salmonella (NTS), predominantly serovars Typhimurium and Enteritidis, are leading causes of bloodstream infections that can manifest as severe invasive disease with high case fatality often seen in young children and human immunodeficiency virus (HIV)-infected individuals (4, 5). The global burden of enteric fever and invasive nontyphoidal Salmonella (INTS) disease in 2021 was estimated to be 9.8 million cases leading to 169,300 deaths and 12.8 million disability-adjusted life years (3). In addition to causing invasive infections, nontyphoidal Salmonella are responsible for a substantial burden of gastroenteritis worldwide with an estimated incidence of 93.8 million cases annually (6).

Antimicrobial resistance poses a considerable barrier to treating severe Salmonella spp. infections (7), and alternative interventions such as vaccination would reduce the reliance on antimicrobial therapies. While several typhoid vaccines have been licensed, there are none available against S. Paratyphi A, S. Typhimurium, or S. Enteritidis. Multivalent Salmonella vaccine candidates have entered clinical trials, including bivalent options for INTS disease or enteric fever as well as trivalent INTS/typhoid vaccines (8); however, only one vaccine candidate with the potential to cover all four serovars of concern has been described and is in pre-clinical development (9). Effective broad-spectrum Salmonella vaccine strategies are therefore urgently needed and would simplify vaccine development for salmonellosis from a global perspective.

Lipopolysaccharide (LPS) is an important virulence factor for GNB and is composed of three structural domains: lipid A (endotoxin), the core oligosaccharide, and the hypervariable O-polysaccharide. In examining 25 different S. enterica strains, Kauffman found that while the O-polysaccharides differed with each of the strains, they all had the same five sugars in their core (10). This observation led to the hypothesis that Salmonella spp. Lipopolysaccharide has a common core to which specific O-polysaccharides were attached. The inner core sugars exhibit very limited structural diversity across Enterobacteriaceae, and thus this region represents a potential broad-spectrum vaccine target. Early investigators reasoned that antibodies directed against core may cross-react with the lipopolysaccharide of many other GNB and consequently be broadly protective (11).

To generate core-specific antibodies, GNB mutants were developed that lacked the immunodominant O-polysaccharide to expose the core. Heat-killed bacteria of a Rc (J5) mutant of E. coli O111:B4 and a Re mutant of Salmonella Minnesota were used as killed whole cell vaccines and studied extensively in pre-clinical models of GNB infection and later administered to human subjects (12, 13). Pre- and post-vaccination sera following immunization of healthy subjects with the J5 mutant of E. coli O111:B4 was evaluated in a large, randomized multicenter trial of patients bacteremic with a wide range of GNB. Compared to patients who received the pre-immune sera, there was significantly improved survival among patients administered the immune sera, including those who required pressors for the treatment of shock (14). However, further development of this killed bacterial vaccine was not pursued.

A subunit vaccine was developed comprised of detoxified lipooligosaccharide (LOS) from the J5 mutant of E. coli that was non-covalently complexed with a group B Neisseria meningitidis outer membrane protein (OMP) (15), referred to as J5 dLOS/OMP. Extensive pre-clinical testing with this vaccine demonstrated that both active and passive immunization strategies were protective in various pre-clinical models of experimental Gram-negative sepsis including intra-abdominal sepsis, burn wound infection, pneumonia, and neutropenia (16-19). Furthermore, administration of J5 dLOS/OMP to healthy subjects in two phase 1 clinical trials was found to be immunogenic, safe, and well-tolerated (20, 21).

Early studies with monoclonal antibodies (mAbs) elicited by immunization with the E. coli J5 mutant demonstrated the potential for E. coli core-specific antibodies to broadly recognize LPS from various heterologous GNB, including laboratory strains of S. enterica (22). These findings suggest that an E. coli J5 core-based vaccine could elicit heterologous protective immunity against Salmonella spp.; however, direct experimental evidence in support of this hypothesis is lacking.

Thus, there is a need in the art for immunization methods against Salmonella species. Particularly, the art is deficient in methods for immunizing against Salmonella species with a vaccine cross-reactive with the lipopolysaccharide conserved inner cores in Salmonella. The present invention fulfills this long-standing need and desire in the art.

### SUMMARY OF THE INVENTION

The present invention is directed to a method for immunizing against a Salmonella species in a subject. In this method, a vaccine that broadly targets the lipopolysaccharide inner core antigens in gram negative bacteria is administered to the subject. A related method further comprises administering a Salmonella species-specific vaccine to the subject, thereby increasing protection against an antimicrobial resistant Salmonella species.

The present invention is further directed to a method for reducing the likelihood of or preventing an onset of a salmonella infection in a subject. In the method a J5 dLOS/OMP vaccine effective to cross-react with the lipopolysaccharide inner core in a Salmonella species is administered to the subject. A related method further comprises administering a Salmonella species-specific vaccine to the subject to increase protection against an antimicrobial resistant Salmonella species.

The present invention is directed further to a method for immunizing against an antimicrobial resistant Salmonella species in a subject. In this method, a J5 dLOS/OMP vaccine effective to elicit a cross-reactive antibody response against the lipopolysaccharide inner core in a Salmonella species is administered to the subject. A Salmonella species-specific vaccine effective to elicit a different antibody response against the Salmonella species is administered.

Other and further aspects, features, benefits, and advantages of the present invention will be apparent from the following description of the presently preferred embodiments of the invention given for the purpose of disclosure.

### BRIEF DESCRIPTION OF THE DRAWINGS

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1B show that J5 dLOS/OMP vaccination elicits E. coli LOS-specific IgG responses that cross-react with Salmonella LPS. FIG. 1A: A New Zealand white rabbit was vaccinated three times with J5 dLOS/OMP co-formulated in Hiltonol. Baseline (pre) and post-vaccination (post) serum IgG titers against E. coli J5 LOS were measured by ELISA. FIG. 1B: Crude LPS from protein-digested bacterial lysates was separated and probed with the J5 dLOS/OMP antisera. Lane 1: S. paratyphi A ATCC9150; Lane 2: S. Paratyphi B CMF 6999; Lane 3: S. typhimurium D65; Lane 4: S. paratyphi C I8; Lane 5: Staphylococcus aureus USA 300 (negative control); Lane 6: S. Newport Chile 361; Lane 7: S. enteritidis R11; Lane 8: S. typhi Ty2; Lane 9: E. coli J5. The table lists each type of bacteria and their respective Salmonella serogroup, if applicable.

FIG. 2 shows that J5 dLOS/OMP-induced IgG recognizes epitopes within the Salmonella core oligosaccharide. J5 dLOS/OMP rabbit antisera were immunoblotted against crude LPS extracts from protein-digested Salmonella Minnesota mutants of rough and deep rough phenotype. Lane 1: smooth parent (SF1111); Lane 2: Ra mutant (SF1112); Lane 3: Rb mutant (SF1127); Lane 4: RcP+mutant (SF1195); Lane 5: Rd1P+mutant (SF1286); Lane 6: Rd1P-mutant (SF1121); Lane 7: Rd mutant (SF1117); Lane 8: Re mutant (SF1167); Lane 9: E. coli J5 (Rc mutant). The table lists each bacteria and their respective core oligosaccharide phenotype.

FIGS. 3A-3B show that E. coli J5 LOS does not inhibit binding of LPS-specific antibodies raised against an S. enteritidis O-antigen based conjugate vaccine. A competitive ELISA was performed to determine the specificity of S. enteritidis COPS:FliC-induced IgG with the vaccine homologous LPS after co-incubation with various doses of either S. enteritidis R11 LPS (FIG. 3A) or E. coli J5 LOS (FIG. 3B). Bars represent the mean±SD from two separate experiments. Statistical comparisons were made with the no LPS/LOS conditions using a one-way ANOVA with Dunnett's multiple comparisons test. *, P≤0.05; ****, P≤0.0001; ns, not significant.

FIGS. 4A-4B show that passive transfer of J5 dLOS/OMP antisera protects mice against heterologous challenge with nontyphoidal Salmonella. Naïve CD-1 mice were intraperitoneally (IP) administered PBS (n=10, grey squares) or a 1:25 dilution of rabbit sera from baseline (Pre, n=15, black circles) or post-vaccination (Post, n=15, white circles). Treated mice were then IP challenged 4-6 hours later with either (FIG. 4A) 1×10⁶ CFU of S. enteritidis R11 or (FIG. 4B) 6.8×10⁵ CFU of S. typhimurium D65 and monitored daily for 20-21 days. Kaplan-Meier survival curves were compared by log-rank test. **, P≤0.01; ***, P≤0.001.

FIGS. 5A-5C show that active immunization with J5 dLOS/OMP elicits protective immunity against lethal systemic challenges with S. enteritidis or S. typhimurium. Groups of 20 mice were immunized three times with PBS (black circles) or J5 dLOS/OMP (white circles). (FIG. 5A) Serum anti-J5 LOS IgG responses were measured in a subset (n=10 mice/group). Bars represent the geometric mean titer, and groups were compared with a Mann-Whitney U test. Immunized mice were IP challenged 4-6 weeks later with either (FIG. 5B) 9.7×10⁵ CFU of S. enteritidis R11 or (FIG. 5C) 6.9×10⁵ CFU of S. typhimurium D65 and monitored daily for 30 days. Kaplan-Meier survival curves were compared by log-rank test. **, P≤0.01; ****, P≤ 0.0001.

FIGS. 6A-6B show in vivo functional activity of J5 dLOS/OMP-induced antibodies in mouse models of bacteremia and endotoxemia which serves to reduce bacterial burden and reduce endotoxin lethality. FIG. 6A: Groups of 5 mice were treated IP with of PBS (grey squares) or a 1:20 dilution of rabbit sera from baseline (black circles) or post-vaccination (white circles). Mice were challenged intravenously 2 h later with 2×10⁴ CFU of S. enteritidis R11. Bacterial loads in the spleen were determined 21 h after challenge. The median burden is represented by the bar. FIG. 6B: Groups of 10 mice were immunized with three doses of PBS (black circles) or J5 dLOS/OMP+Hiltonol (white circles). On day 7 post-vaccination, mice were sensitized to endotoxin by treatment with D-GaIN (20 mg) and simultaneously challenged with E. coli 0111: B4 LPS (5 ng). Kaplan-Meier survival curves were compared by log-rank test. ****, P≤0.0001; ns, not significant.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, the terms “consist of” and “consisting of” are used in the exclusive, closed sense, meaning that additional elements may not be included.

As used herein, the term “includes” or “including” refers to “including, but not limited to”. The terms “includes, “including” and “including, but not limited to” are used interchangeably.

As used herein, the term “about” refers to a numeric value, including, for example, whole numbers, fractions, and percentages, whether or not explicitly indicated. The term “about” generally refers to a range of numerical values (e.g., +5-10% of the recited value) that one of ordinary skill in the art would consider equivalent to the recited value (e.g., having the same function or result). In some instances, the term “about” may include numerical values that are rounded to the nearest significant figure.

As used herein, the term “subject” refers to a human or a non-human mammal, for example, a farm animal, a food animal or a companion animal.

In one embodiment of the present invention, there is provided a method for immunizing against a Salmonella species in a subject, comprising administering to the subject a vaccine that broadly targets the lipopolysaccharide inner core antigens in gram negative bacteria. Further to this embodiment, the method comprises administering a Salmonella-specific vaccine to the subject, thereby increasing protection against antimicrobial resistant Salmonella species.

In both embodiments, the subject may be a human or a non-human mammal. In both embodiments, the vaccine may be an Escherichia coli J5 vaccine. Particularly the Escherichia coli J5 vaccine is a J5 dLOS/OMP vaccine. In addition the J5 dLOS/OMP vaccine may elicit cross-reactive antibody responses with the lipopolysaccharide inner core antigens of the Salmonella species. Furthermore the J5 dLOS/OMP vaccine may elicit cross-protective immunity against an infection caused by a non-typhoidal Salmonella species.

In one aspect of both embodiments, the Salmonella species is a typhoidal serovar that is at least one of a Salmonella typhi, a Salmonella paratyphi A, a Salmonella paratyphi B, or Salmonella paratyphi C. In another aspect, the Salmonella species is a non-typhoidal serovar that is at least one of a Salmonella typhimurium, a Salmonella Newport, a Salmonella enteritidis, or a Salmonella Minnesota or a mutant thereof.

In another embodiment of the present invention, there is provided a method for reducing the likelihood of or preventing an onset of a salmonella infection in a subject, comprising administering to the subject a J5 dLOS/OMP vaccine effective to cross-react with the lipopolysaccharide inner core in a Salmonella species. Further to this embodiment, the method comprises administering to the subject a Salmonella species-specific vaccine to increase protection against an antimicrobial resistant salmonella infection.

In one aspect of both embodiments, the salmonella infection is salmonellosis. In this aspect, the Salmonella species may be a Salmonella typhimurium, a Salmonella Newport, a Salmonella enteritidis, or a Salmonella Minnesota. Also in this aspect, the subject may be a human, a farm animal or a companion animal.

In another aspect of both embodiments, the salmonella infection is typhoid fever or paratyphoid fever. In this aspect, the the Salmonella species may be a Salmonella typhi, a Salmonella paratyphi A, a Salmonella paratyphi B, or Salmonella paratyphi C. Also in this aspect, the subject is human.

In yet another embodiment of the present invention, there is provided a method for immunizing against an antimicrobial resistant Salmonella species in a subject, comprising administering to the subject a J5 dLOS/OMP vaccine effective to elicit a cross-reactive antibody response against the lipopolysaccharide inner core in a Salmonella species; and administering a Salmonella species-specific vaccine effective to elicit a different antibody response against the Salmonella species.

In this embodiment, the Salmonella species is Salmonella typhi, a Salmonella paratyphi A, a Salmonella paratyphi B, or Salmonella paratyphi C, Salmonella typhimurium, a Salmonella Newport, a Salmonella enteritidis, or a Salmonella Minnesota. Also in this embodiment, the subject may be a human or a non-human mammal.

Provided herein are methods for immunizing a subject against a Salmonella species, for example, typhoidal and non-typhoidal Salmonella serovars, both including antimicrobial resistant Salmonella species. Particularly, the Escherichia coli J5 dLOS/OMP vaccine, which targets the inner core region of lipopolysaccharide, elicits cross-reactive antibody responses against the Salmonella species. Moreover, the J5 dLOS/OMP vaccine elicits cross-protective immunity against non-typhoidal salmonella infections.

The typhoidal Salmonella species are serovars of Salmonella typhi and Salmonella parathyroid A, B or C. The non-typhoidal Salmonella species are serovars of Salmonella typhimurium, Salmonella Newport, Salmonella enteritidis, or Salmonella Minnesota or mutants thereof.

Also provided are methods for reducing the likelihood of acquiring or of the onset of a salmonella infection, including the prevention thereof. Examples of a salmonella infection caused by typhoidal Salmonella species are typhoid fever and paratyphoid fever. An example of a salmonella infection caused by non-typhoidal Salmonella is salmonellosis, such as bacteremia, gastroenteritis and endotoxemia. In addition, the present invention provides a method for immunizing a subject against an antimicrobial resistant Salmonella species, both typhoidal species and non-typhoidal species. Complementing the J5 dLOS/OMP vaccine with a Salmonella species-specific vaccine adds the ability to elicit an antibody response different from that of the J5 dLOS/OMP vaccine.

The subjects receiving the benefit of immunization are generally mammalian, such as human subjects and non-human mammals, for example, farm animals, including food animals, and companion animals. All subjects generally may be susceptible to non-thyroidal salmonella. Human subjects may be susceptible to thyroidal salmonella.

The following examples are given for the purpose of illustrating various embodiments of the invention and are not meant to limit the present invention in any fashion.

### Example 1

### Method & Materials

### Bacterial Strains, Medium, and Growth Conditions

The strains used herein are listed in Table 1 and were maintained in Hi-Soy (HS) media (5 g/L sodium chloride (MilliporeSigma, Burlington, MA), 10 g/L soytone (Teknova, Hollister, CA), 5 g/L Hy-yest (Kerry Bio-Science, Beloit, WI)). For inoculum verification and bacterial load determination, bacteria were plated onto either HS agar or tryptic soy agar (TSA) (MilliporeSigma).

TABLE 1
Bacterial strains used herein

| Strain | Description | Source |
|---|---|---|
| S. paratyphi A ATCC | Reference strain | American Type |
| 9150 | | Culture Collection |
| | | (ATCC) |
| S. paratyphi B sensu | Clinical isolate from Chile | (39) |
| stricto CMF 6999 | | |
| S. typhimurium D65 | Clinical isolate from Mali | (40) |
| S. paratyphi C I8 | Clinical isolate from Mali | (41) |
| S. Newport Chile 361 | Clinical isolate from Chile | (41) |
| S. enteritidis R11 | Clinical isolate from Mali | (40) |
| S. typhi Ty2 | Reference strain | ATCC (42) |
| S. Minnesota | | (27) |
| SF1111 | Smooth parent strain | |
| SF1112 | Ra mutant | |
| SF1127 | Rb mutant | |
| SF1195 | RcP⁺ mutant | |
| SF1286 | Rd₁P⁺ mutant | |
| SF1121 | Rd₁P⁻ mutant | |
| SF1117 | Rd₂ mutant | |
| SF1167 | Re mutant | |
| Staphylococcus aureus | Methicillin-resistant | ATCC |
| clone USA300 | strain | |
| Escherichia coli J5 | Rc mutant of E. coli | (12) |
| | O111: B4 | |

### Immunization and Sera Collection

All animal experiments were approved by the Institutional Animal Care and Use Committee at the University of Maryland Baltimore or Cocalico Biologicals (Stevens, PA).

### Mouse Immunization

Six to eight-week old female CD-1 mice (Charles River Laboratories, Wilmington, MA) were immunized intraperitoneally on days 0, 14, and 28 with either PBS, J5 dLOS/OMP (50 μg polysaccharide/dose) or J5 dLOS/OMP (22.5 μg polysaccharide/dose) admixed with Hiltonol® (poly-ICLC, 25 μg/dose) (Oncovir, Inc, Washington, DC). Vaccines were diluted in PBS and sterile filtered prior to delivery. Control groups received sterile PBS. Sera were collected three weeks after the final immunization.

### Rabbit Immunization

A female New Zealand White rabbit was immunized intramuscularly with J5 dLOS/OMP (10 μg polysaccharide/dose) formulated with Hiltonol® (6.25 μg/dose) on days 0, 14, and 28 (Cocalico Biologicals). Sera were collected prior to the first immunization and day 14 after the final dose. All serum samples were stored at −80° C. until analysis.

Challenge Studies after Active Immunization

Bacteremia model: Mice were challenged with 9.7×10⁵ CFU of S. enteritidis R11 or 6.9×10⁵ CFU of S. typhimurium D65 delivered intraperitoneally 4-6 weeks following the third dose. Bacteria were harvested from lawns grown overnight at 37° C. on HS agar.

Endotoxemia model: One week after the final immunization, mice were treated intraperitoneally with 20 mg of D-galactosamine (D-GaIN, MilliporeSigma), to sensitize them to the biological effects of endotoxin, and simultaneously challenged with 5 ng of purified E. coli O111:B4 LOS (List Biological Laboratories, Campbell, CA) (23). Survival was monitored daily up to 30 days. Animals that were overtly moribund were immediately euthanized and recorded as having died.

### Passive Transfer and Challenge Studies

Baseline and day 14 post-vaccination rabbit sera were heat-inactivated at 56° C. for 30 min and diluted in sterile PBS. In one experiment, 6-8-week-old female CD-1 mice were injected intraperitoneally with 200 μL of PBS or a 1:25 dilution of rabbit sera. Four to six hours later, they were challenged intraperitoneally with 1×10⁶ CFU of S. enteritidis R11 or 6.8×10⁵ CFU of S. typhimurium D65. Mice were monitored daily for 20-21 days after challenge, recording weight loss and mortality. In another experiment, 8-10-week-old female CD-1 mice were treated intraperitoneally with 400 μL of PBS or a 1:20 dilution of rabbit sera. Two hours later, mice were infected intravenously with 2×10⁴ CFU of S. enteritidis R11 grown to early log phase (˜3 h) in HS broth. Twenty-one hours after infection, spleens were harvested, weighed, and mechanically disrupted with a tissue homogenizer (Omni International, Kennesaw, GA). The bacterial suspension was serially diluted, and bacterial loads (CFU/g tissue) were determined through drop plating onto TSA (5 μL drops in triplicate).

### ELISA for Titrating Vaccine-Induced IgG Responses

Ninety six-well microtiter plates (Greiner Bio-One, Monroe, NC) were pre-treated with 2 μg/mL of poly-L-lysine (MilliporeSigma) in PBS (1 h, 37° C.). Following washes, plates were coated with 2-5 μg/mL of E. coli J5 LOS (List Biological Laboratories) in PBS (3 h, 37° C.). Plates were washed and blocked with 10% Omniblok non-fat dry milk (AmericanBio, Canton, MA) in PBS (overnight, 4° C.). Mouse or rabbit sera samples were serially diluted in blocking buffer+0.05% Tween-20 (MilliporeSigma) using 3-fold dilutions and added to the blocked ELISA plates in duplicate wells (1 h, 37° C.). After washing, horseradish peroxidase-conjugated goat anti-mouse IgG or anti-rabbit IgG (Jackson ImmunoResearch Laboratories, West Grove, PA) diluted 1:2,000 in blocking buffer+0.05% Tween-20 was added (1 h, 37° C.). Plates were washed, and bound antibodies were detected with 1-Step Ultra TMB substrate solution (Thermo Fisher Scientific, Waltham, MA) for 15 min at room temperature. The reaction was stopped with 2 N sulfuric acid (Thermo Fisher Scientific), and the absorbance at 450 nm was measured using a SpectraMax Plus 384 spectrophotometer (Molecular Devices, San Jose, CA). Endpoint titers were expressed as ELISA units (EU) and calculated by multiplying the lowest absorbance value above 0.25 by the dilution factor yielding that absorbance value.

### Competitive ELISA

Rabbit antisera raised against an S. enteritidis O-antigen based glycoconjugate vaccine (COPS:FliC (24)) was diluted 1:3,000 and incubated with different concentrations of either S. enteritidis R11 LPS or E. coli J5 LOS (100-0.01 μg/ml). S. Enteritidis R11 LPS was isolated following hot/phenol water extraction as described previously (25). The antisera/LPS mixture was added to 96-well microtiter plates coated with S. enteritidis R11 LPS, and bound anti-COPS:FliC antibodies were detected by ELISA as described above.

### Western Blot Analyses of Lipopolysaccharide

Overnight bacterial cultures were normalized to an OD₆₀₀ of 1. Either 2 mL (for E. coli J5 or the Salmonella clinical isolates) or 0.2 mL (for the Salmonella SF series) was lysed in 200 μL of Tricine SDS sample buffer (Thermo Fisher Scientific)+5% β-mercaptoethanol, heated at 100° C., and treated with 100 μg/lysate of proteinase K (Qiagen, Germantown, MD) to digest proteins (26). LPS extracts were separated on 10-20% Tricine gels (Thermo Fisher Scientific) and wet-transferred to PVDF membranes. After blocking with 3% bovine serum albumin (MilliporeSigma) in PBS+0.1% Tween-20, membranes were probed with J5 dLOS/OMP rabbit antisera (1:1,000, incubated overnight at 4° C.) followed by 0.05 μg/mL of goat anti-rabbit IgG-HRP (SeraCare, Gaithersburg, MD) for 1 h at room temperature. Blots were developed using SuperSignal West Pico PLUS Chemiluminescent substrate (Thermo Fisher Scientific). Bands were visualized using the Chemi-Doc MP system (Bio-Rad, Hercules, CA).

### Statistical Analysis

Kaplan-Meier survival curves were compared using a log-rank test. Bacterial burdens were compared using either a two-tailed, Mann-Whitney U test (for unpaired comparisons between PBS and pre-immune groups) or a two-tailed, Wilcoxon matched-pairs signed rank test (for paired comparisons between pre-immune and post-vaccination groups). Serum antibody titers between J5 dLOS/OMP vaccinated and PBS control mice were compared using a two-tailed, Mann-Whitney U test. Absorbance values in the competitive ELISA were compared using a one-way analysis of variance followed by Dunnett's multiple comparisons test. P-values≤0.05 were considered significant. All analyses were performed in GraphPad Prism v6.

### Example 2

### Results

Cross-Reactivity of J5 Antisera with Nontyphoidal and Typhoidal Salmonella

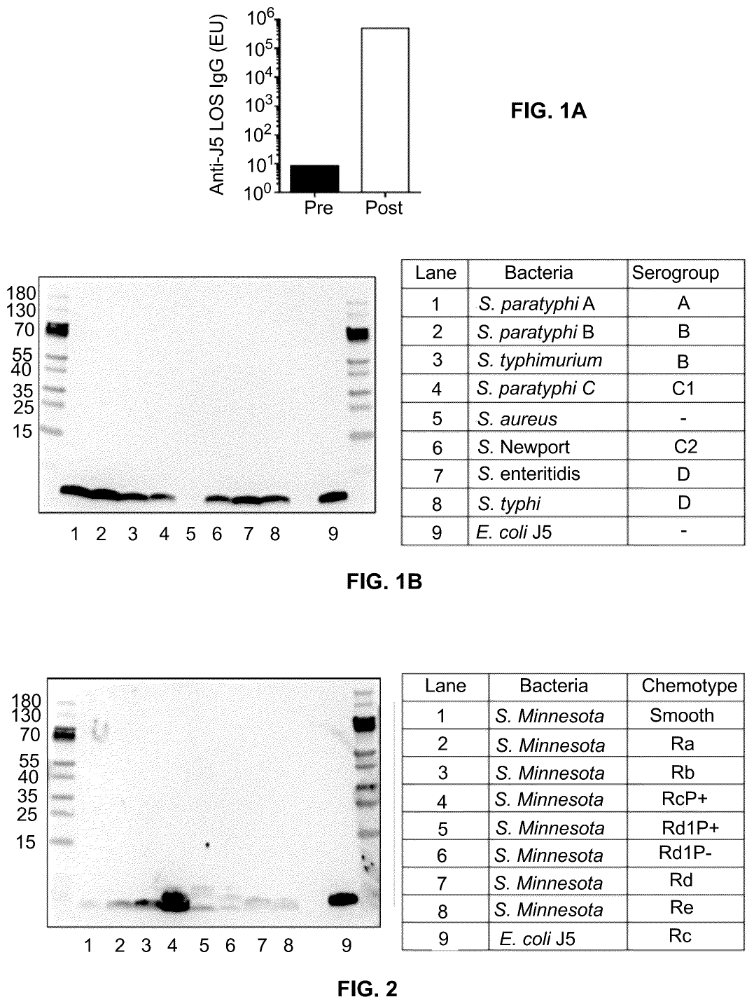

To demonstrate that vaccination with E. coli J5 dLOS/OMP, abbreviated hereafter as J5, elicits cross-reactive antibodies against Salmonella. A New Zealand White rabbit was immunized with three doses of the J5 vaccine, which mounted a robust serum antibody response after vaccination (anti-LOS IgG titer of 5.1×10⁵ EU) (FIG. 1A), which is consistent with previous observations (11). The post-vaccination rabbit sera produced a single, low-molecular-weight band in Western blotting experiments with proteinase-K digested E. coli J5 lysate, which corresponds to a truncated LPS molecule consisting of lipid A and the inner core oligosaccharide (FIG. 1B, lane 9). The J5 antisera was also tested for cross-reactivity with different strains of Salmonella enterica representing typhoidal and nontyphoidal serovars belonging to serogroups A-D and which are commonly implicated in human illness. It was found that J5-induced IgG strongly recognized LPS from serovars paratyphi A, B, and C, Newport, typhimurium, enteritidis and typhi, forming a single, high-mobility band similar to E. coli J5 LOS (lanes 1-4, 6-8). J5-induced IgG did not react with lysate from the Gram-positive Staphylococcus aureus (lane 5).

J5 Vaccine-Induced IgG Binds to Epitopes within the Salmonella Core Oligosaccharide

A series of Salmonella rough mutants was utilized that are defective in core biosynthesis and express stepwise truncations in core chain length (decreasing in size from Ra to Re) to determine which epitopes on the core oligosaccharide contribute to anti-J5 antibody binding (27). Immunoblotting with bacterial lysates revealed that J5-induced IgG recognizes, to varying degrees, all rough chemotypes as well as the smooth parent strain (FIG. 2, lane 1), demonstrating the greatest intensity for RcP+LOS (lane 4), which is the same chemotype as E. coli J5 (lane 9).

Specificity of Salmonella LPS-Specific IgG Elicited by Immunization with J5 or S. enteritidis O-Antigen Based Conjugate Vaccines

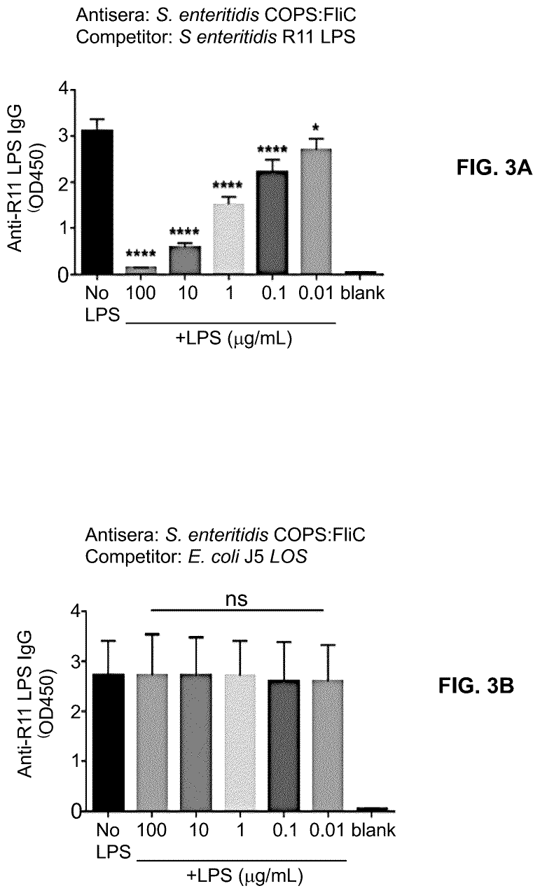

The immunogenicity and efficacy of S. enteritidis and S. typhimurium O-antigen based glycoconjugate vaccines (COPS:FliC) in rabbits was previously reported (24). To determine if the J5 antisera recognized a similar epitope on LPS as the conjugate-induced antibodies, a competitive ELISA was performed using S. enteritidis R11 LPS as the capture antigen, which is homologous to the core and O-polysaccharide in S. enteritidis COPS:FliC. The conjugate antisera displayed strong reactivity with its homologous LPS as expected, and the addition of unconjugated R11 LPS inhibited the binding of these antisera to S. enteritidis LPS in a dose-dependent manner (FIG. 3A). By contrast, J5 LOS was unable to disrupt the binding of the S. enteritidis COPS:FliC antisera at any concentration tested (FIG. 3B).

Cross-Protective Efficacy of J5 Immunization Against Systemic Challenge with NTS Bloodstream Isolates

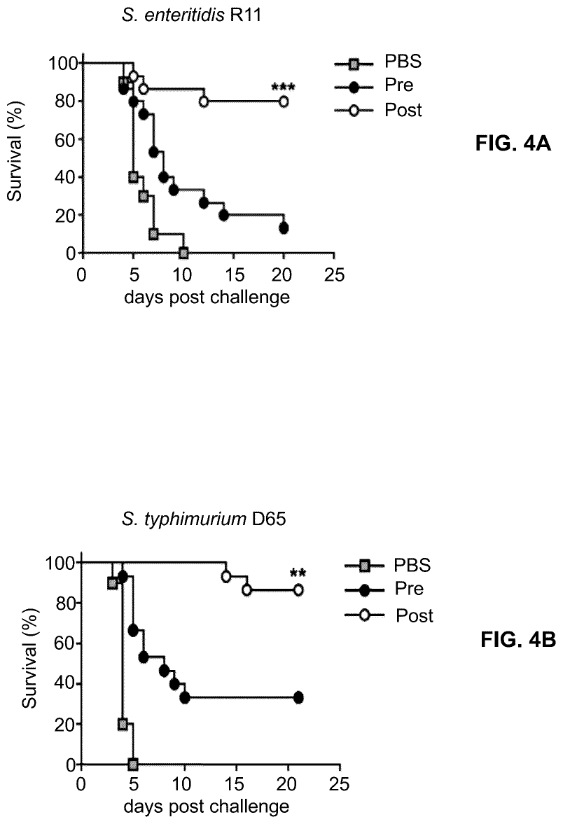

Whether the cross-reactive immunity elicited by J5 immunization would confer protection against a heterologous Salmonella challenge was evaluated. Naïve outbred CD-1 mice were administered baseline or post-vaccination sera from the J5-immunized rabbit, or PBS as a negative control, and then challenged intraperitoneally (IP) with 1×10⁶ CFU of S. enteritidis R11 (FIG. 4A). The challenge resulted in ≥86.7% mortality in mice given PBS or baseline rabbit sera, whereas only 20% of the mice treated with the J5 post-vaccination sera died (P≤0.001). Similarly, passive transfer of the J5 antisera demonstrated a significant protective effect against IP challenge with 6.8×10⁵ CFU of S. typhimurium D65 relative to the pre-vaccination group and PBS controls (P≤0.01) (FIG. 4B).

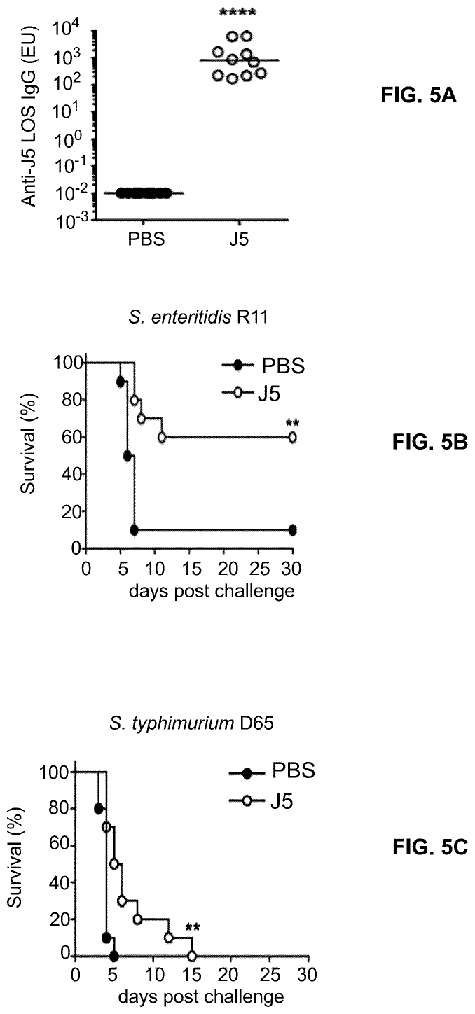

CD-1 mice were immunized with 3 doses of either J5 or the vaccine diluent (PBS) to determine the efficacy of active vaccination against systemic challenge with NTS (n=20/group). J5 immunization stimulated robust serum anti-LOS IgG titers (geometric mean titer (GMT)=816.9 EU) compared to control animals where vaccine-induced antibody responses were negligible (FIG. 5A). Half of the mice were then challenged IP with 9.7×10⁵ CFU of S. enteritidis R11. While most of the PBS control mice succumbed rapidly within 7 days, the J5-immunized mice were significantly protected from mortality (P≤0.01) (FIG. 5B). The remaining mice were challenged IP with 6.9×10⁵ CFU of S. typhimurium D65. Survival was prolonged in vaccinated mice compared to controls with a mean time-to-death of 3.9 and 6.9 days in PBS and J5 groups, respectively (P<0.01) (FIG. 5C).

### Functional Activity of J5 Vaccine-Induced Immunity In Vivo

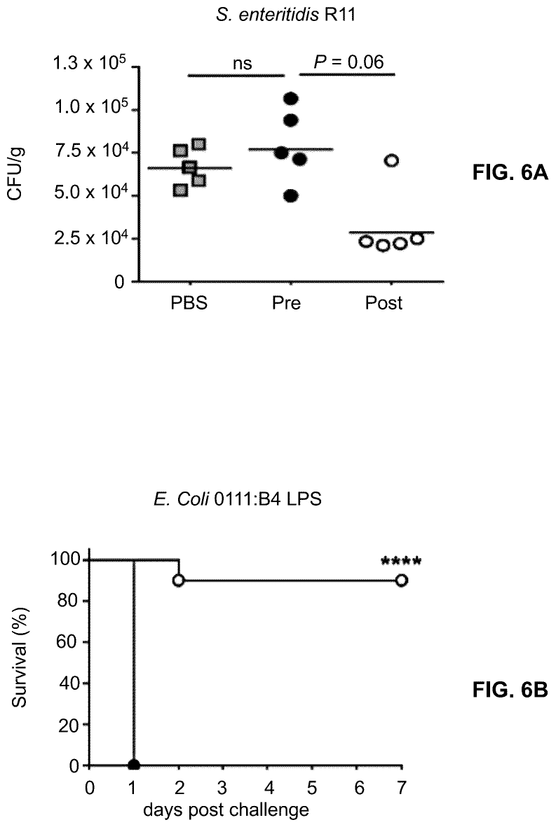

J5-induced antibodies with cross-reactivity to Salmonella may play an important role in protection. To test this, naïve CD-1 mice were passively administered either rabbit pre-immune or J5 immune sera and then challenged them intravenously with 2×10⁴ CFU of S. enteritidis R11 (FIG. 6A). Control animals received PBS in place of sera. After 21 hours, S. enteritidis was readily detectable in the spleens of mice treated with PBS or pre-immune rabbit sera (median recovered bacteria=6.7-7.5×10⁴ CFU/g). Prophylaxis with post-vaccination rabbit antisera prior to challenge trended toward reduced bacterial loads at this time point, but this did not achieve significance (2.4×10⁴ CFU/g, P=0.06).

The endotoxic activity of LPS is attributable to its lipid A component, which can lead to exuberant innate immune activation and septic shock. Therefore, the in-vivo endotoxin neutralization potential of J5 vaccination was determined using the D-galactosamine (D-GaIN) sensitization mouse model (23) and E. coli LPS, which shares a similar hex-acylated lipid A structure with S. enterica (28). CD-1 mice were immunized three times with either J5 or PBS. Upon D-GaIN treatment, the J5 vaccinated mice were significantly protected from lethal challenge with LPS extracted from E. coli O111:B4 compared to PBS controls (P<0.0001) (FIG. 6B).

### DISCUSSION

Salmonella infections are responsible for a significant burden of disease in both resource limited and industrialized nations. The continued spread of antimicrobial resistant Salmonella necessitates greater investment into prophylactic interventions, such as vaccination. The present invention demonstrates the applicability of a candidate E. coli inner core-based vaccine (J5 dLOS/OMP) against heterologous Salmonella infection. J5 vaccine-induced antibodies recognize the LPS from several typhoidal and nontyphoidal serovars of Salmonella. Challenge studies in mice demonstrated that J5 immunization protected animals against lethality caused by S. enteritidis and S. typhimurium infection. Protection was associated with an ability of anti-J5 antibodies to mitigate lethal LPS-induced endotoxemia in mice and possibly promote in vivo clearance of bacteria.

Following exposure to GNB or LPS, the serotype-specific O-antigen is immunodominant and constitutes a well-appreciated target of protective antibodies (11). For Salmonella, protection afforded by LPS-specific immunity could extend to other serovars bearing structurally similar O-antigen, i.e., of the same serogroup; however, support for heterologous serogroup protection is mixed. As an example, immunization of mice with live-attenuated S. typhimurium strains or outer membrane vesicles derived from S. typhimurium bearing smooth LPS (group B with O-antigen determinants 1,4,(5),12) was reported to cross-protect against lethal challenge with S. choleraesuis (group C₁; O: 6,7) or S. enteritidis (group D; O: 1,9,12) in some studies (29-31), but not others (32-34). Data to support cross-protective immunity in humans are limited. Immunization with the licensed live oral S. typhi vaccine (Ty21a) elicits cross-reactive cellular and humoral immune responses against S. paratyphi A and B (35); however, there are limited data suggesting that Ty21a may confer protection against S. paratyphi B, but not S. paratyphi A (36, 37). Prior exposure to S. typhi in controlled human infection models fails to protect against bacteremia and enteric fever caused by S. paratyphi A and vice versa (38). Further, typhoid vaccines based on the Vi capsular polysaccharide would not afford protection against disease caused by Vi-negative strains of S. typhi or other Salmonella serovars lacking Vi that may emerge with widespread use of these vaccines. Hence, targeting conserved antigens, such as the core oligosaccharide of LPS, may enable the development of broad-spectrum Salmonella vaccines.

The present invention demonstrated that antibodies induced by J5 vaccination recognized LPS from a collection of typhoidal and nontyphoidal Salmonella. J5 dLOS/OMP-induced IgG antibodies bound most intensely to RcP+LPS from S. Minnesota (as was also the case with the LPS core-specific mAb WN1 222-5 (22), which was expected as the S. Minnesota RcP+mutant bears the same core chemotype as the vaccine parent strain (E. coli J5). By contrast, anti-J5 antibodies reacted relatively weakly with core structures that were more complete than RcP+ and with full-length LPS. The binding capabilities of inner core-specific antibodies to Salmonella LPS could potentially be influenced by steric effects from the outer core and/or O-antigen. Furthermore, the specificity of protective anti-LPS antibodies generated by J5 immunization may differ from that of traditional Salmonella O-antigen-based conjugate vaccines.

Despite several reports of core-specific mAbs and core LPS-based vaccines, the protective efficacy of core-specific antibodies against invasive Salmonella infections was unknown (11). The present invention demonstrated that parenteral immunization of mice with J5 improved survival against systemic challenge with NTS. Antibody responses elicited by J5 immunization may play an important protective role. Although the underlying mechanism is still unclear, this protection data corroborate other preclinical studies with the J5 vaccine where active and passive immunization protected against several different gram negative bacteria including Pseudomonas aeruginosa, Klebsiella pneumoniae, and E. coli (11). J5 vaccination should also protect against the typhoidal serovars typhi and paratyphi A and other nontyphoidal serovars of clinical interest.

J5 antibodies may mediate protection against Salmonella in part by limiting endotoxin release into circulation or promoting its clearance, similar to previous findings in a neutropenic rat model of lethal P. aeruginosa sepsis (25). The anti-inner core mAb WN1 222-5 was also shown to reduce LPS-induced fever in rabbits and lethality in D-GaIN sensitized mice upon passive transfer, but not against Salmonella bacteria (22). It is also possible that anti-J5 antibodies mediate direct bactericidal activity, perhaps through opsonophagocytic killing, which might limit the ability of Salmonella to cause bacteremia and colonize tissues. Further work to uncover the mechanisms by which anti-core antibodies mediate Salmonella clearance and protection is warranted.

In conclusion, evidence is provided that the E. coli J5 dLOS/OMP vaccine, which targets the highly conserved inner core region of LPS, elicits cross-reactive antibody responses against Salmonella and cross-protective immunity against NTS infection. If efficacy is confirmed against both typhoidal and nontyphoidal serovars, then targeting the core oligosaccharide determinant of LPS may hold potential as a serovar-agnostic vaccine strategy against salmonellosis. It also is contemplated that the J5 vaccine could complement licensed S. typhi vaccines or other Salmonella vaccines in clinical development by adding an additional protective antigen, independent of O-polysaccharides, that exhibits broad coverage. This could conceivably mitigate the risk of outbreaks caused by non-vaccine serovars, including those resistant to antimicrobials.

### REFERENCES

1. Marks et al., Lancet Glob Health 2017, 5: e310-e23.
2. Garrett et al., Lancet Glob Health 2022, 10: e978-e88.
3. (IHME) IfHMaE. Global Burden of Disease 2021: Findings from the GBD 2021 Study. Seattle, WA: IHME, 2024.
4. Gilchrist et al., EcoSal Plus 2019, 8.
5. Marchello et al., Lancet Infect Dis 2022, 22:692-705.
6. Majowicz et al., Clin Infect Dis 2010, 50:882-9.
7. Antimicrobial Resistance C. Global burden of bacterial antimicrobial resistance in 2019: a systematic analysis. Lancet 2022, 399:629-55.
8. MacLennan et al., Open Forum Infect Dis 2023,10: S58-S66.
9. Boerth et al., Vaccines (Basel) 2023, 11.
10. Kauffmann et al., Zentralbl Bakteriol 1961, 182:57-66.
11. Cross A S. et al., Microbiol Mol Biol Rev 2023, e0004522.
12. Ziegler et al., J Immunol 1973, 111:433-8.
13. DeMaria et al., J Infect Dis 1988, 158:301-11.
14. Ziegler et al., N Engl J Med 1982,307:1225-30.
15. Bhattacharjee A et al., J Infect Dis 1996, 173:1157-63.
16. Opal et al., J Infect Dis 2005, 192:2074-80.
17. Neely et al., J Burn Care Rehabil 2002, 23:333-40.
18. Chen et al., Innate Immun 2008, 14:269-78.
19. Cross et al., J Infect Dis 2001, 183:1079-86.
20. Cross et al., Vaccine 2015,33:6719-26.
21. Cross et al., Vaccine 2003, 21:4576-87.
22. Di Padova et al., Infect Immun 1993, 61:3863-72.
23. Galanos et al., Proc Natl Acad Sci USA 1979; 76:5939-43.
24. Baliban et al., Molecules 2018, 23.
25. Cross et al., Innate Immun 2021, 27:266-74.
26. Baliban et al., PLOS Negl Trop Dis 2017, 11: e0005493.
27. Luderitz et al., Bacteriol Rev 1966, 30:192-255.
28. Muller-Loennies et al., Int J Med Microbiol 2007, 297:321-40.
29. Collins F M., J Bacteriol 1968,95:1343-9.
30. Liu et al., Sci Rep 2016, 6:34776.
31. Chen et al., Front Microbiol 2020, 11:588952.
32. Hormaeche et al., Vaccine 1996, 14:251-9.
33. Li P, et al., Front Immunol 2017, 8:1647.
34. Li et al., PLOS Negl Trop Dis 2022, 16: e0010866.
35. Amicizia et al., J Prev Med Hyg 2017, 58: E1-E8.
36. Simanjuntak et al., Lancet 1991, 338:1055-9.
37. Levine et al., Lancet 1987, 1:1049-52.
38. Gibani et al., PLOS Negl Trop Dis 2020, 14: e0008783.
39. Higginson et al., mSphere 2018, 3.
40. Tapia et al., Clin Infect Dis 2015, 61 Suppl 4: S332-8.
41. Fuche et al., Am J Trop Med Hyg 2018, 98:589-94.
42. Morello et al., J Infect Dis 1965, 115:131-41.

What is claimed:

1. A method for immunizing against a Salmonella species in a subject, comprising:
administering to the subject a vaccine that broadly targets the lipopolysaccharide inner core antigens in gram negative bacteria.

2. The method of claim 1, further comprising administering a Salmonella-specific vaccine to the subject, thereby increasing protection against antimicrobial resistant Salmonella species.

3. The method of claim 1, wherein the vaccine is an Escherichia coli J5 vaccine.

4. The method of claim 3, wherein the Escherichia coli J5 vaccine is a J5 dLOS/OMP vaccine.

5. The method of claim 4, wherein the J5 dLOS/OMP vaccine elicits cross-reactive antibody responses with the lipopolysaccharide inner core antigens of the Salmonella species.

6. The method of claim 1, wherein the Salmonella species is a typhoidal serovar that is at least one of a Salmonella typhi, a Salmonella paratyphi A, a Salmonella paratyphi B, or Salmonella paratyphi C.

7. The method of claim 1, wherein the Salmonella species is a non-typhoidal serovar that is at least one of a Salmonella typhimurium, a Salmonella Newport, a Salmonella enteritidis, or a Salmonella Minnesota or a mutant thereof.

8. The method of claim 4, wherein the J5 dLOS/OMP vaccine elicits cross-protective immunity against an infection caused by a non-typhoidal Salmonella species.

9. The method of claim 1, wherein the subject is a human or a non-human mammal.

10. A method for reducing the likelihood of or preventing an onset of a salmonella infection in a subject, comprising:
administering to the subject a J5 dLOS/OMP vaccine effective to cross-react with the lipopolysaccharide inner core in a Salmonella species.

11. The method of claim 10, further comprising administering to the subject a Salmonella species-specific vaccine to increase protection against an antimicrobial resistant salmonella infection.

12. The method of claim 10, wherein the salmonella infection is salmonellosis.

13. The method of claim 12, wherein the Salmonella species is a Salmonella typhimurium, a Salmonella Newport, a Salmonella enteritidis, or a Salmonella Minnesota.

14. The method of claim 12, wherein the subject is a human, a farm animal or a companion animal.

15. The method of claim 10, wherein the salmonella infection is typhoid fever or paratyphoid fever.

16. The method of claim 15, wherein the Salmonella species is a Salmonella typhi, a Salmonella paratyphi A, a Salmonella paratyphi B, or Salmonella paratyphi C.

17. The method of claim 15, wherein the subject is a human.

18. A method for immunizing against an antimicrobial resistant Salmonella species in a subject, comprising:
administering to the subject a J5 dLOS/OMP vaccine effective to elicit a cross-reactive antibody response against the lipopolysaccharide inner core in a Salmonella species; and
administering a Salmonella species-specific vaccine effective to elicit a different antibody response against the Salmonella species.

19. The method of claim 18, wherein the Salmonella species is Salmonella typhi, a Salmonella paratyphi A, a Salmonella paratyphi B, or Salmonella paratyphi C, Salmonella typhimurium, a Salmonella Newport, a Salmonella enteritidis, or a Salmonella Minnesota.

20. The method of claim 18, wherein the subject is a human or a non-human mammal.
